(19) United States
(12) Patent Application Publication
(10) Pub. No.: US 2009/0111093 A1
(43) Pub. Date: Apr. 30, 2009

(54) Methods and compositions for pre-symptomatic or post-symptomatic diagnosis of alzheimer's disease and other neurodegenerative disorders

(75) Inventors: Douglas C. Wallace, Irvine, CA (US); Pinar E. Coskun, Long Beach, CA (US)

Correspondence Address: STOUT, UXA, BUYAN & MULLINS LLP, 4 VENTURE, SUITE 300, IRVINE, CA, 92618, US

(73) Assignee: The Regents of the University of California

(21) Appl. No.: 10/594,825

(22) PCT Filed: Mar. 29, 2005
(86) PCT No.: PCT/US05/10266

§ 371 Date: Aug. 22, 2008

Related U.S. Application Data
(60) Provisional application No. 60/557,612, filed on Mar. 29, 2004.

(51) Int. Cl. C12Q 1/68

(52) U.S. Cl. 435/6

(57) ABSTRACT

Methods, compositions and apparatus (e.g., test kits, test systems, reagents, related computer software, calculators, etc.) for pre-symptomatic or post-symptomatic diagnosis of Alzheimer's Disease or other disorders associated with the formation of β-amyloid deposits (e.g., plaques) and/or β-amyloid fibrils. Also, methods, compositions and apparatus assessing the efficacy of treatments for such disorders. Sample cells, tissue or body fluid are obtained from a human or animal subject and analyzed to determine whether or to what extent certain mitochondrial DNA control region (mtDNA CR). Significantly elevated numbers of these mtDNA CR mutations may indicate that the subject suffers from, or is at increased risk for development of, Alzheimer's Disease or other disorders associated with the formation of β-amyloid deposits (e.g., plaques) and/or β-amyloid fibrils. A significant decrease in the numbers of these mtDNA CR mutations during treatment for the disorder may indicate that the treatment is effective.

### RELATED APPLICATION

This application claims priority to U.S. Provisional Patent Application No. 60/557,612 entitled “Methods and Apparatus for Determining Mitochondrial Control Region Mutations Associated With Alzheimer's Disease” filed on Mar. 29, 2004, the entirety of which is expressly incorporated herein by reference.

### STATEMENT REGARDING GOVERNMENT SPONSORED RESEARCH

This invention was made with Government support under Grant Nos. AG13154 and NS21328, awarded by the National Institutes of Health. The Government has certain rights in this invention.

### FIELD OF THE INVENTION

This invention relates generally to molecular biology and medicine, and more particularly to methods and compositions usable for diagnosis and prognostication in patients who suffer from, or ar at risk for development of, Alzeheimer's Disease or other neurodegenerative disorders.

### BACKGROUND OF THE INVENTION

Many of the normal physiological functions of the mammalian body come about, at least in part, through the ability of proteins to body adopt various sequence-dependent structures. However, sometimes protein sequences form aberrant, misfolded, insoluble aggregates known as amyloid fibrils. These amyloid fibrils are thought to be involved in the pathogenesis of various amyloid diseases of genetic, infectious and/or spontaneous origin, including but not limited to Alzheimer's disease, spongiform encephalopathies, Parkinson's disease, type II diabetes, Creutzfeldt-Jakob disease, Down's Syndrome-associated dementia, Huntington's disease, macular degeneration, various prion diseases and numerous others. In at least some of these amyloid diseases, amyloid fibrils lead to the development of amyloid plaques.

Alzheimer's Disease is a progressive neurodegenerative disease and is the most common form of progressive dementia observed in the elderly. It is associated with the accumulation of β-amyloid (Aβ) plaques and neuritic tangles in the brain. However, the cause of Alzheimer's Disease remains largely unknown.

Mitochondrial abnormalities have frequently been observed in Alzheimer's Disease brains and deficiencies in OXPHOS enzymes have been reported in Alzheimer's Disease patient brains and systemically. Certain germline mutations of mitochondrial DNA (mtDNA) have also been associated with certain Alzheimer's Disease patients of European descent. These include a tRNA^{Gln} gene mutation at nucleotide pair (np) 4336, found in about 5% of late-onset patients, and a ND1 np 3397 mutation, which converts a highly conserved methionine to a valine. The association between the np 4336 variant and Alzheimer's Disease has been confirmed in three out of four independent European studies. Alzheimer's Disease has been further linked to germline mtDNA variation in reports that European mtDNA lineages (haplogroups) J and Uk are protective of Alzheimer's Disease and Parkinson's Disease (PD) and are also associated with increased longevity. Finally, Alzheimer's Disease brains have been observed to have increased somatic mtDNA rearrangement mutations, with the common 5 kilobase (kb) mtDNA deletion being elevated about 15 fold in Alzheimer's Disease patient brains up to age 75 years.

The mtDNA CR is a 1000 nucleotide pair (np), non-coding, region of the mtDNA that contains the promoters for the initiation of heavy (H) and L-strand transcription (PH & PL), the associated mitochondrial transcription factor (mtTFA) binding sites, the three conserved sequence blocks (CSB) I-III, and the origins of H-strand replication (OH). Hence, the CR is the primary site for the regulation of mtDNA transcription and replication.

The mtDNA codes for 13 essential OXPHOS polypeptides, 22 tRNA genes, and a 12S and 16S rRNA gene, in addition, the mtDNA CR encompasses the light (L)- and heavy (H)-strand promoters (P_{L} and P_{H}); their mitochondrial transcription factor A (mtTFA) binding sites; the downstream conserved sequence blocks (CSB) I, II, and III; and the origins of H-strand replication (O_{H1} and O_{H2}) Recently, tissue-specific, mtDNA CR mutations have been discovered to accumulate with age. A T414G transversion in the mtTFA binding site of P_{L} accumulates in cultured skin fibroblasts and can be detected at low levels in skeletal muscle, but not in brain, using applicant's sensitive protein nucleic acid (PNA)-clamping polymerase chain reaction (PCR) method. In addition, the A189G and T408A CR mutations accumulate with age in skeletal muscle and a T150C mutation accumulates in white blood cells. However, to date no specific, somatic, mtDNA CR mutations have been reported for normal or AD patient brains. However, specific mtDNA CR mutations have been found to accumulate with age in particular tissues. For example, a T to G transversion at np 414 (T414G) was found to accumulate with age in human skin fibroblasts (Michikawa et al, 1999, Science 286:774-779) and an A189G and a T408A mutation were observed to accumulate in skeletal muscle (Wang et al, 2001, PNAS 98:4022-4027). However, the T414G mutation could not be detected in normal brain using a sensitive protein nucleic acid (PNA)-clamping polymerase chain reaction (PCR) technique (Murdock et al, 2002, NAR 28:4350-4355).

### SUMMARY OF THE INVENTION

The present invention provides methods, compositions and apparatus (e.g., test kits, test systems, reagents, related computer software, calculators, etc.) for pre-symptomatic or post-symptomatic diagnosis of neurodegenerative disorders associated with the formation of β-amyloid deposits (e.g., plaques) and/or β-amyloid fibrils by determining whether or to what extent mtDNA CR mutations are present in tissue or cells of the subjects body.

In accordance with the invention, there is provided a method wherein sample cells are obtained from a human or animal subject, DNA is extracted from the sample cells and the DNA is subjected to mitochondrial DNA control region amplification. Thereafter, a determination is made whether nomoplasmic 414 and 477 nucleotide variants are present. If 414 and 477 nucleotide variants are deemd to be present, the mutant molecules are cloned and sequenced to confirm the mutation. The number of such mutations may then be compared to that of a relevant control group or population. If the number of such mutations is significantly greater than control, it may be concluded that the subject has developed or is at risk to develop a neurodegenrative disorder or other β-amyloid disorder (e.g., macular degeneration).

Further in accordance with the invention, there are provided methods for determining the efficacy or non-efficacy of treatments for neurodegenrative disorder or other β-amyloid disorders (e.g., macular degeneration) by determining changes in the quantity or severity of mtDNA CR mutations.

Further aspects and details of this invention will become apparent to those of skill in the art upon reading the detailed description and examples set forth herebelow.

### BRIEF DESCRIPTION OF THE DRAWINGS

FIGS. 1A and 1B show the somatic mtDNA CR mutation distribution in AD and control brains. FIG. 1A is a schematic representation of nps 16000-570 of the mtDNA CR. The numbers below the line mark the mtDNA nps, and the boxes above the line represent the regulatory elements. The thick horizontal lines below the CR map represent the locations of the AD (red), control (blue), or common (gold) heteroplasmic mutations. FIG. 1B is a table showing the number of heteroplasmic mutations in mtDNA CR regulatory elements in AD and control brains.

FIGS. 2A-E show the results of a PNA-clamping PCR assay for T414G mtDNA mutation in AD and control brains. FIG. 2A shows the agarose gel results of controls. FIG. 2A shows the agarose gel results of AD patients. The individual samples in a and b are identified by the age of the subject. Two PCRs are shown for each subject, one in the absence (I) and the other in the presence (+) of a PNA encompassing the 414 wildtype base, which suppresses amplification of the wild-type mtDNA. FIG. 2C shows Fokl digestion of the PNA-clamping PCR products confirming the presence of the T414G mutation from AD brains. FIG. 2C shows Fokl digestion of the PNA-clamping PCR products confirming the presence of the T414G mutation from AD and control brains run into same gel for comparison. Lanes in c are labeled with age of AD patients. −c, the Fokl digestion result from the PCR product from wild-type plasmid; +c, the result from a T414G mutant plasmid. The arrow indicates the T414G Fokl product. FIG. 2E is a graphic showing sequence analysis of CR fragments from a 74-year-old subject. The 414 region was PNA-clamping PCR-amplified, the resulting fragments were reamplified without PNA, and the final fragments were cloned and sequenced. The mutant nucleotide G (indicated with an arrow) is seen in three of five clones.

FIGS. 3A and 3B are bar graphs showing the total number of heteroplasmic mtDNA CR mutations observed by cloning and sequencing CR clones from AD and control brain samples. FIG. 3A shows the number of mutants from all age groups (range 59-94); *, P<0.01. FIG. 3B shows the number of mutants from three different age groups: 59-69, 7079, and 80 & up; for 80 & up DNA mutation frequency, **, P<0.001.

FIGS. 4 A-D are graphs showing the specific somatic mtDNA CR mutants and their percentage of heteroplasmy in AD and control brains, Subjects are listed by age. FIG. 4A shows CR nps 1-100 data from control (normal) brains. FIG. 4B shows CR nps 1-100 data from the brains of patients having AD. FIG. 4C shows CR nps 101-570 data from control (normal) brains. FIG. 4B shows CR nps 101-570 data from the brains of patients having AD. The specific mutations are listed below the abscissa lines. The percentage of each mutation in each individual's brain is given by the height of the bar of that color. Homoplasmic germ-line mutations were also observed for these mutations. For the np 1-100 region, A73G was seen in six ADs and four controls. In the np 101-570 region, T146C was seen in three ADs and two controls; T152C was seen in three ADs and four controls; A189G was seen in no ADs and one control; and T195C was seen in two ADs and two controls. T414C and T477C were not found in the homoplasmic state in either AD or control samples.

FIG. 5 is a bar graph comparing the incidence of T414G transversion mutation in brains from AD, DS, ADPD, PD and control (normal) subjects. The number of frontal cortex samples assayed in each group is displayed beneath the X axis.

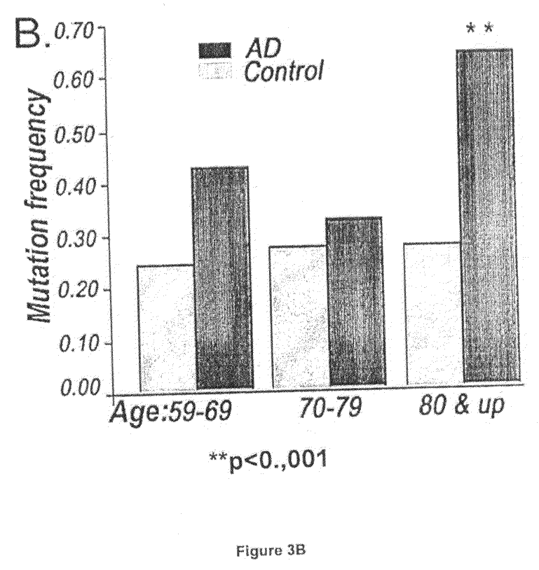

FIG. 6 is a bar graph showing mtDNA CR Somatic mtDNA mutation frequency in demented DS, AD and control brains. The demented DS brains were found to have a 61% increase in the number of mtDNA CR mutations, relative to controls; while AD brains had a 76% increase.

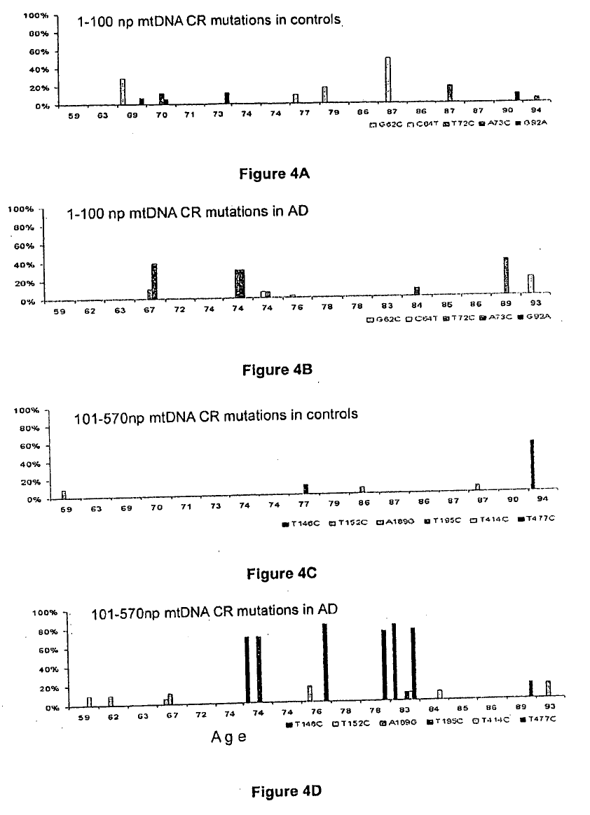

FIG. 7 is a table showing the distribution of demented DS, AD and control somatic mtDNA mutations within the mtDNACR regulatory elements.

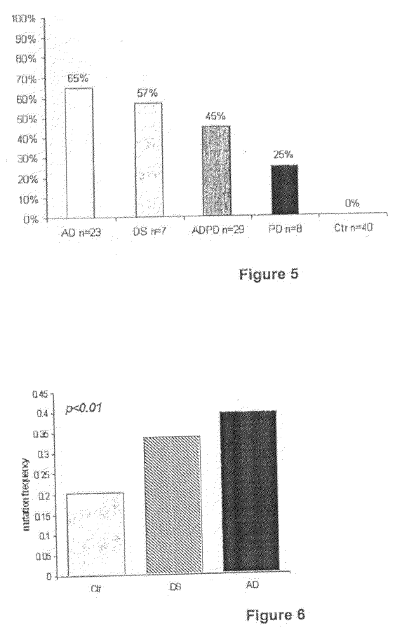

FIG. 8 is a bar graph showing mtRNA level (L-Strand/H-strand) in DS, AD and control brains. This graph is representing the ratio of transcription levels of Light-strand (ND6 mRNA level) over Heavy strand (ND2 mRNA level) in the 40 to 74 years of control, AD and DS patient brains. This graph displays significant reduction of mtRNA level of L-Strand/H-strand about 50% in AD and DS cases.

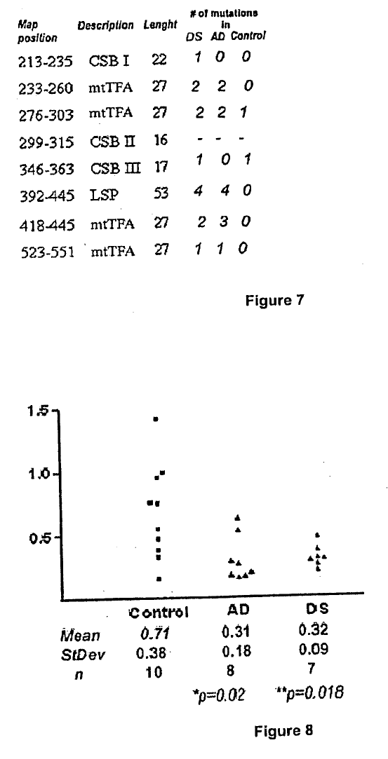

### DETAILED DESCRIPTION AND EXAMPLES

The following detailed description and examples, the accompanying drawings and the above-set-forth brief description of the drawings are intended to describe and illustrate certain examples of the invention only and shall not be construed as limiting the scope of the invention in any way.

Applicants have discovered that, in the brains of patients who suffer from Alzheimer's Disease, there is an increased incidence of mutations located in the mtDNA CR within elements known to be involved in mtDNA L-strand transcription and H-strand replication. Moreover, these mutations are associated with a reduction in the mtDNA, L-strand, ND6 mRNA and in the mtDNA copy number. Hence, it is concluded that somatic mtDNA mutations are a common feature of sporadic AD, and that they can account for the observed mitochondrial dysfunction.

As described in Example 1 below, applicants have tested for the T414G mutation by PNA-clamping PCR in AD brain frontal cortex. This PNA-clamping PCR is described here below and in Murdock D G and Wallace D C; PNA-mediated PCR Clamping. Applications and Methods. Methods Mol Biol. 208:145-64 (2002). This method can be used to test for any of the mtDNA CR in accordance with this invention.

In those tests it was found that 65% of the AD brains were positive for this mutation while none of the normal control brains had the mutation. To investigate this phenomenon further, applicants cloned and sequenced multiple CR clones from the brains of AD patients and age-matched controls. This revealed that the AD brains had a 63% overall increase in CR mutations, and these mutations were preferentially located in sequence motifs in the CR that were known to be involved in regulating mtDNA transcription and regulation. For example, applicants have found seven mutations each in the CSBI and in the PH & PL mtTFA elements in AD brains but none in the control brains. Moreover, the age distribution of the AD CR mutations was distinctive, being 65% higher than controls in the ages 59-69, 14% higher in ages 70-79 brains, and 130% higher in ages 80 and older AD brains.

Beyond the overall increase in mtDNA CR somatic mutations in AD brains, applicants also discovered two CR mutations that were unique to the brains of AD patients, the T414C and T477C mutations. In addition, mutations at T146C, T152C, A189G, and T195C were more common in AD brains than controls. Finally, the T146C, T195C and T477C mutations increased to very high levels in the AD brains, in certain cases coming to represent 70-80% of all of the mtDNAs in the patient's brain. Moreover, these mutations were often found together in AD brains, but not in control brains. Finally, these specific mtDNA CR mutations were found at very high frequencies primarily in patients in the age range of 70 to 83 years old, the same range that had the reduced frequency of more random CR mutations. This implies that there are two classes of AD. In one case, a few CR mutations arise early in development, become widely disseminated throughout the brain, and then clonally amplified in each cell giving rise to an earlier onset dementia associated with a high frequency of a few mutations in the brain. In the other case, the mutations accumulate later in development so that each individual mutation is confined to a fewer number of cells. When these mutations are clonally amplified within their respective cells, then each mutation can only come to represent a few percent of the total mtDNA CR mutations in the brain.

In either case, when the percentage of the mutant mtDNAs reached a high enough level within a cell, it inhibits mtDNA transcription and/or replication. This leads to reduced L-strand transcription, inhibition of mtDNA replication, respiratory deficiency, premature neuronal death and dementia. Hence, these data indicate that somatic mtDNA CR region mutations are the cause of late-onset AD. Therefore, detection of these mutations becomes and excellent tool to confirm the diagnosis of AD or to predict those individuals who are at risk for the disease though currently pre-symptomatically.

Applicants have also evaluated Alzheimer and Parkinson disease patient (ADPD) brains for the hypothesis of increased mtDNA CR mutations. First, T414G mutation has been tested on this set of brain samples. Interestingly, 52% of the patient brains were observed to be positive for this mutation. Other CR mutations have been still under investigation for ADPD brains.

Down Syndrome (DS) patients also develop a senile dementia associated with amyloid plaques and neurofibrillary tangles analogous to that of AD, but at a much younger age. Hence, if mtDNA CR mutations were an important for developing dementia in association with plaques and tangles, then it may be reasonably predicted that the brain of demented DS patients should have comparable diversity and density of somatic mtDNA mutations as AD patient brains, but at a younger age.

To determine if this was true, applicants used a PNA-clamping PCR technique to test frontal cortex DNA from demented AD brain samples for the T414G CR mutation. Consistent with the above-noted AD brain results, it was determined that 57% of the demented AD brains, ages 40 to 62, harbored the T414G mutations as compared to 65% of AD brains, ages 59-93, but none of the control brains, ages 59-94.

To determine if the brains of demented DS patients also harbored other mtDNA CR region mutations in important functional elements, applicants PCR amplified, cloned, and sequenced the CR of the frontal cortex mtDNAs from 7 DS brains with dementia, and compared the results with those of our AD and control subjects. The demented DS brains were found to have a 61% increase in the number of mtDNA CR mutations, relative to controls; while AD brains had a 76% increase. Furthermore, the demented DS CR mutations were concentrated in known mtDNA transcription and replication regulatory elements, just as found for AD.

Finally, as in the case of the AD brains, the demented DS brains had a 50% reduction in the L-strand ND6 mRNA levels. The mean ratio for the DS brains was 0.32±0.09, while that for the controls was 0.71±0.38, P=0.018. Thus, the accumulation of plaques and tangles is associated with increased mitochondrial somatic mtDNA mutations and decreased mtDNA transcripts.

### EXAMPLE 1

(Identification of mtDNA CR Mutations Associated With Amyloid Disorders)

Brain Samples from AD Patients and Controls

Frontal cortex brain samples from age-matched AD and control cadaveric subjects were used in these experiments. A total of 23 AD and 40 control (non-AD) brain samples were pathologically confirmed and used in this study. The mtDNA hypervariable region (np-16000-100) of each brain sample was sequenced and those samples belonging to the European mtDNA haplogroups H, U, J and T were chosen for further cloning and sequencing studies to minimize the polymorphic differences common for intercontinental comparisons. To eliminate the possibility that the observed CR variants were the product of the spurious amplification of nuclear DNA (nDNA)-encoded, mtDNA pseudogenes, all PCR protocols were applied to cells lacking mtDNA (ρ° cells) to assure that no mtDNA-like sequences could be amplified.

### Detection of the T414G Mutation in AD Brains

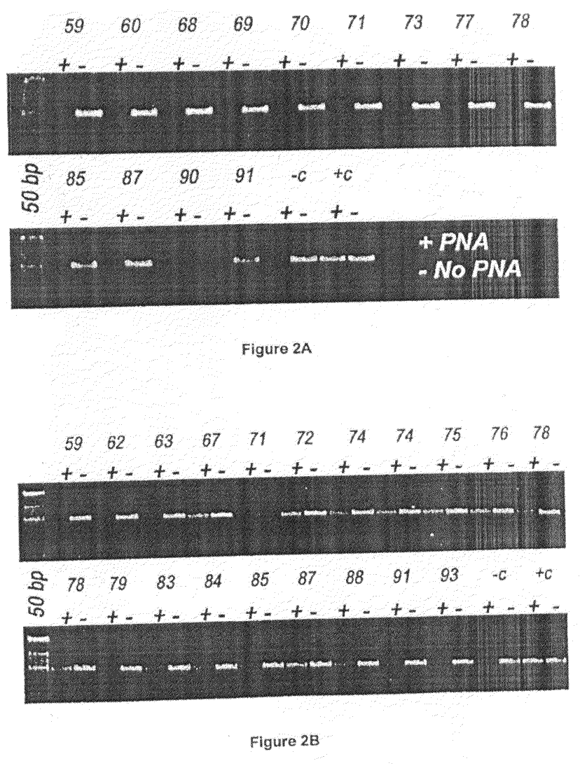

The T414G mutation was sought in the frontal cortex DNAs by the PNA-clamping PCR procedure shown in FIG. 2A, which can detect one mutant mtDNA in 1000 wild-type molecules. The presence of the T414G mutation in the resulting 334 np PCR product was confirmed by cleavage with Fokl and by cloning and sequencing individual PCR molecules, as shown in FIGS. 2C and 2E.

Identification of Novel mtDNA CR Mutations in AD Brains by Cloning and Sequencing

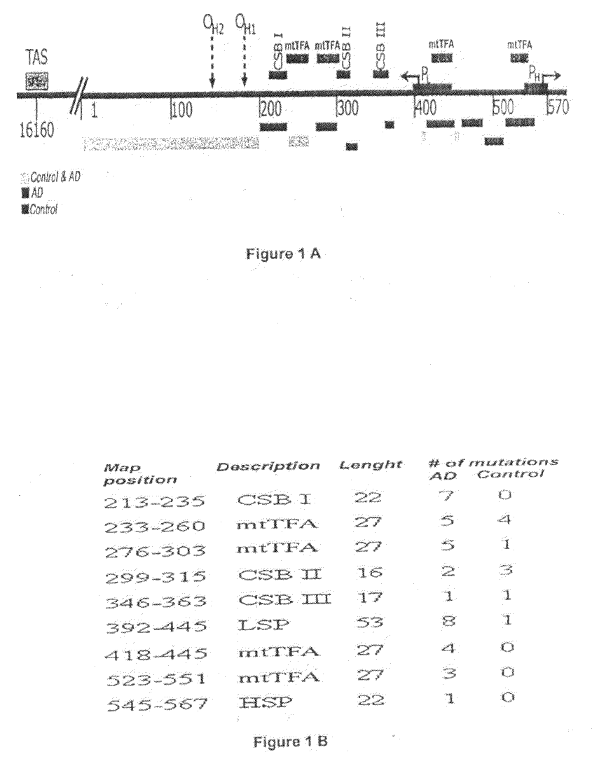

Additional somatic mtDNA CR mutations were identified by PCR-amplification of the mtDNA CR between nucleotide pairs (nps) 16527 and 636, cloning and sequencing as shown in FIG. 1. Frontal cortex genomic DNA was extracted using the pure gene kit (Gentra system) and the CR amplified using the primers np 16527-16546 (5′-CCT AAA TAG CCC ACA CGT TC-3′) and np 617-636 (5′-TGA TGT GAG CCC GTC TAA AC-3′) together with high fidelity Epicentre failsafe Taq DNA polymerase. The desired PCR fragments were purified by agarose gel electrophoresis, extracted using the NucleoTrap gel kit (Clontech), cloned using the TOPO TA cloning protocol (Invitrogen), and the desired plasmids purified by the mini-preparation. Plasmid DNAs were cycle sequenced using BigDye dideoxy chain terminator chemistry (Applied Biosystem) on an ABI 3100 capillary sequencer, with the sequencing results analyzed using “Sequencer v4.0.5” (Gene Code Corporation).

Quantification of mtDNA Transcript Levels and Copy Number

To determine the ratio of mtDNA L-strand to H-strand transcripts, total RNA was extracted from the cortex tissue using TRIZOL (Gibco-BRL system) and the L-strand, ND6, mRNA and H-strand, ND2, mRNAs were reverse transcribed and quantified by quantitative real time (qRT)-PCR. ND6 was amplified using forward primer np 14260-14279 (5′-ATC CTC CCG AAT GAA CCC TG-3′) and reverse primer np 14466-14485 (5′-GAT GGT TGT CTT TGG ATA TA-3′). ND2 mRNA was amplified using the using the same primes as employed to determine the mtDNA/nDNA ratio²⁵.

The relative mtDNA/nDNA ratio was determined by qRT-PCR of genomic DNA. The mtDNA primers were in the ND2 gene and the nDNA primers were in the 18S rRNA.

### RESULTS

### The T414G CR Mutation is Found in AD Brains but not Controls

When the presence of the T414G mutation was analyzed in the frontal cortex genomic DNAs of all 23 AD and 40 control brains by PNA-clamping PCR, 65% of the AD brains tested positive for the T414G mutation, but none of the controls (FIGS. 2A & B). The presence of the T414G mutation was confirmed in the AD samples by Fokl restriction endonuclease digestion and by direct cloning and sequencing (FIGS. 2C, 2D & 2E).

### Identification and Quantification of AD Brain CR Mutations

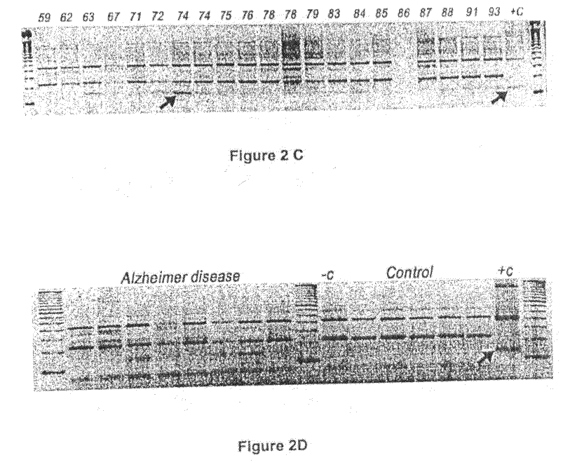

To determine the generality of the increased presence of somatic mtDNA CR mutations in AD brains, we PCR amplified, cloned and sequenced 10 to 20 CR clones from each of 16 AD and 17 control brain samples, giving a total of 250 AD and 235 control clones analyzed. As shown in FIG. 3A, this revealed an overall 63% increase in the frequency of heteroplasmic mtDNA CR mutations in AD brains versus controls (P<0.01). Moreover, as seen in FIG. 3B, division of the AD cases into decade age groups revealed that the 59-69 year age group had a 79% increase, the 70-79 year age group had a 18% increase, while the 80 and older age group had a 130% in mtDNA CR mutations relative to controls, with the difference between the 80 and older AD patients and controls being highly significant (P<0.001).

To determine the functional significance of these CR mutations, the positions of the mutations were correlated with that of the known functional elements of the mtDNA CR. Again with reference to FIG. 1, no clear difference in mutation distribution was seen between AD and control samples in the CR between nps 1 to 100 where few regulatory elements have been identified. By contrast, a significant increase in CR mutations was seen in the AD brain clones in the region between nps 101 and 570, which encompasses most of the known mtDNA regulatory elements.

Moreover, the AD mutants, but not the control mutants, were preferentially located in known functional transcription and replication elements. For example, as seen in FIG. 1B, seven heteroplasmic, CR mutations were observed in AD brains in CSBI, but none were seen in controls. Likewise, as seen in FIG. 2B, 17 heteroplasmic mutations were found in the four mtTFA binding sites (two between P_{L} and P_{H} and two between CSBI and CSBII) in AD brains while only five mutations were observed in the controls (P<0.001). Indeed, seven heteroplasmic mutations were, present in the two mtTFA binding sites associated with P_{H} and P_{L} in AD brains, but none were found in these mtTFA sites in control brains. Therefore, mtDNA CR mutations are more common in AD patient brains and they preferentially affect functionally important motifs.

High Level mtDNA CR Mutant Heteroplasmy in AD Brains

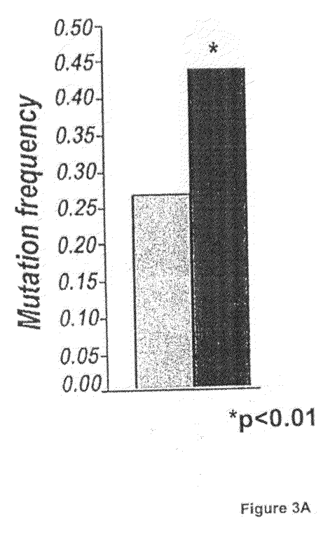

Not only were CR mutations more prevalent in AD brains, they were frequently present at exceptionally high percentages of heteroplasmy as seen in FIG. 4. While no marked differences were found between AD and control brains between nps 1 and 100 (FIG. 4A versus FIG. 4B), multiple high percentage heteroplasmic mutations were found in AD brains relative to controls in the region between np 100 and 570 (FIG. 4C versus FIG. 4D).

Two of the identified higher percentage heteroplasmy, CR mutations proved to be specific for AD brains. One AD mutation, T414C, was found in 59, 83, and 84 year old AD patients at about 10% heteroplasmy, but was not present in any controls. The second AD-specific mutation, T477C, was found in the 76, 78, 83 year old AD patients at 70-80% heteroplasmy and in an 89 year old patient at 20% heteroplasmy, but not in controls (FIGS. 4C & 4D).

Four other high percentage heteroplasmy CR mutations were found predominantly in AD brains, but also in some controls, though at lower levels and later ages. A T146C mutation was found in 74 and 83 year old AD patient brains at 70 to 80% heteroplasmy, but also in one 94 year old control at about 50% heteroplasmy. A T195C mutation was found in 74 and 83 year old AD patients at 80% and 10% heteroplasmy, respectively; but also in one 77 year old control at about 10% mutant. A T152C mutation was found in 67 and 76 year old AD patient brains at 5-20% heteroplasmic, and also in one 87 year old control at 5% heteroplasmy. A A189G mutation was found in 62, 67, and 93 year old AD brains, at 5 to 20% heteroplasmy; but also in 59 and 86 year old control brains at less then 10% (FIG. 4D versus 4C).

These same CR mutants also co-occurred more often in AD brains than in controls. Four AD brains harbored more than one heteroplasmic mutation. The 67 year old AD brain had both the T152C and A189G mutations, though at low percentages heteroplasmy; the 74 year old AD brain had the T146C and T195C mutations at very high levels of heteroplasmy; the 76 year old AD brain harbored the T152C and T477C mutations at lower and higher heteroplasmy, respectively; and the 83 year old AD brain harbored the T146C and T477C mutations at high percentages heteroplasmy as well as the T195C and T4141C mutations at low percent heteroplasmy. None of the control brains harbored more than one heteroplasmic CR mutation. Furthermore, six AD patients were homoplasmic for the T146C mutation, and four of these were also homoplasmic for the T195C mutation. By contrast, only two controls were homoplasmic for the T146C mutation and none of these had the T195C mutation.

Finally, all of the AD patients that harbored individual mtDNA CR mutations with heteroplasmy levels of 70% or greater occurred in the age range of 74 to 83 years (4 of 7 cases or 60%), while no patients were found with a very high level heteroplasmic mutation between ages 59 and 72 and between 84 and 93. Therefore, mtDNA CR mutations are more common, accumulate earlier, and can be present at higher percentages of heteroplasmy in AD patient brains than in control brains.

Reduced mtDNA L-Strand Transcripts and Copy Number in AD Brains

Most of the observed heteroplasmic mtDNA CR mutations observed in AD brains occurred in proximity to P_{L}, from which L-strand transcription is initiated; CSBI, after which the L-strand transcript is cleaved by the MRP-RNase to yield the 3′-OH replication primer; and O_{H1} and O_{H2}, where mtDNA polymerase γ¹⁶ initiates H-strand replication. Therefore, it may reasonably be expected that the CR mutations found in AD brains should reduce L-strand transcription and mtDNA copy number.

A reduction in AD brain L-strand transcription was confirmed by determining the ratio of the L-strand, ND6 mRNA versus the H-strand, ND2 mRNA using qRT-PCR. The ND6/ND2 mRNA ratio of 12 AD brains was 0.29±0.18, but that of 11 controls was 0.67±0.38, a two fold reduction in the ND6 mRNA level (P=0.01). Similarly, analysis of the mtDNA/nDNA ratio by qRT-PCR of the ND2 and 18S rRNA gene copy numbers gave an average ratio of 12±6.9 for 9 AD brains, but 22±18 for 17 control brains, a 50% reduction in mtDNA copy number (P=0.03).

### CONCLUSION

By analyzing the mtDNA CR sequence variation of pathologically confirmed AD and control brain frontal cortices, applicants have discovered that AD brains harbor a high frequency of heteroplasmic mtDNA CR mutations in key elements that regulate mtDNA L-strand transcription and H-strand replication. Consistent with the functional location of these mutations, AD brains have a marked reduction in the L-strand, ND6, mRNA levels and in the cellular mtDNA copy number.

A reduction in the ND6 mRNA would preferentially inhibit respiratory complex I, since ND6 is the only polypeptide encoded by the mtDNA L-strand and is essential for complex I assembly. In addition, defects in L-strand transcription plus mutations in the CSBI and O_{H1} and O_{H2} sequences would reduce the initiation of H-strand replication, thus accounting for the observed mtDNA depletion. The mtDNA depletion would reduce all 13 of the mtDNA-encoded OXPHOS subunits, thus diminishing the enzyme activities of complexes I, III, IV and V. Consequently, the observed mtDNA CR mutations in AD brains could account for the reduction in mitochondrial OXPHOS enzyme activities that have been observed in AD².

Applicants therorize that the somatic mtDNA CR mutations they have discovered to be associated with AD are caused by mitochondrial ROS production. Mitochondria produce most of the cellular ROS as a toxic by-product of OXPHOS. These ROS, in turn, damage mitochondrial proteins and membranes and mutagenize the mtDNA. Hence, individuals with higher rates of mitochondrial ROS production will acquire somatic mtDNA mutations more rapidly and thus be more prone to develop OXPHOS deficiency and AD as they age.

The tendency of OXPHOS to produce ROS is modulated by the density of electrons that are retained on the electron carriers of the mitochondrial electron transport chain (ETC). Chronic inhibition of the ETC by environmental toxins or mildly deleterious mitochondrial gene mutations, such as the tRNA np 4336 and ND1 np 3997 mutations, would increase the density of electrons on the electron carriers, thus facilitating their spurious transfer directly to O₂ to generate superoxide anion (O₂⁻), the first of the ROS. Superoxide anion could then be converted to hydrogen peroxide (H₂O₂) and H₂O₂ converted to hydroxyl radical (OH), the remaining two ROS. By contrast, a more complete oxidation of the ETC, such as would result from a partially uncoupling of OXPHOS, would diminished the electron density on the carriers and thus reduce ROS production and mtDNA mutagenesis.

In this regard, we have shown that mtDNA mutations that cause uncoupling of OXPHOS became established in human populations as people migrated out of tropical Africa into colder northern Eurasia and Siberia. These mtDNA uncoupling mutations increased mitochondrial heat production at the expense of ATP generation, permitting adaptation to the cold. Today, these same mutations are associated with increased longevity and decreased AD and PD. Striking examples of this phenomenon are seen for the European haplogroups J (sub-haplogroups J1 and J2) and haplogroup Uk. Both haplogroups J1 and Uk were independently founded by the same cytochrome b (cytb) mutation at np 14798. Haplogroup J2 was founded by a different cytb mutation at np 15257. The np 14798 and 15257 cytb mutations alter conserved amino acids in the two coenzyme Q₁₀ binding sites of cytb, and thus could affect proton pumping by the complex III Q cycle. These uncoupling cytb mutations would reduce the mitochondrial ETC electron density, ROS production, somatic mtDNA mutations, and synaptic loss by mitochondrially-induced apoptosis.

A somatic mtDNA mutation can arise within a cell at any time during the life of an individual, from the earliest embryonic cells to the terminally-differentiated post-mitotic cells. Mutations that occur early in development would be propagated through subsequent cell divisions and consequently become widely distributed throughout the organs of the body. By contrast, mutations that arise later in development would be confined to proportionally fewer cells. It is well documented that once a deleterious mtDNA mutation comes to reside in a post-mitotic cell, the mutant mtDNA is selectively amplified and ultimately comes to predominate within that cell. Therefore, mtDNA mutants that arise early in brain development would become widely distributed throughout the cells of the brain through cell division and subsequently be selectively amplified in virtually every cell. These would be the mtDNA mutants that came to represent 70% or more of the mtDNAs in the brains of certain AD patients. Since the mtDNA mutations which arose early in development would also be amplified early, these mutants would reach toxic levels in most brain cells early and thus be found in the brains of earlier-onset AD patients. This would explain why the 74-83 year old AD cases had the lowest frequency of heteroplasmic mtDNA mutations (an 18% increase over controls) (FIG. 3B), but often had one or more mutants at 70-80% percentage heteroplasmy (FIGS. 4C & D).

By contrast in AD cases where the somatic mutations arose late in development, each individual mutant would be confined to a smaller number of the post-mitotic brain cells. If a larger number of these mutations occurred and each was amplified within the cell in which it resided, then these brains would come to have many more mutant mtDNAs, each at a lower overall percentage heteroplasmy. Since these somatic mtDNA mutations arise later in development and more independent mutations would be required to impair sufficient cells to give dementia, the mutations would become amplified to toxic levels later and thus give rise to patients with a later onset disease. This scenario would explain why the 80 and older AD patient brains had a high frequency of different heteroplasmic mtDNA CR mutations (130% over controls) (FIG. 3B), but with none of the mutations representing more than 20% of the mtDNAs of the brain (FIGS. 4C & D). This discontinuity in frequency of mutant mtDNA genotypes in AD brains of different ages was previously observed for the common 5 kb mtDNA deletion. This deletion was found to be increased 15 fold over controls in the brains AD patients who died before age 75, but was ⅕ the control level for brains of AD patients who died after age 75.

Irrespective of how the individual brain cells develop high levels of mtDNA CR mutations, all of the AD patients end up with reduced ND6 mRNA and reduced mtDNA copy number. Once these mutant mtDNAs exceed the expression threshold of the cell, OXPHOS is inhibited, ROS production is increased, the mtPTP becomes sensitized, and the synaptic connections are lost.

This mitochondrial hypothesis can now account for the influence of ApoE alleles on predisposition to AD and the role of Aβ in the AD disease process. Individuals that harbor the ApoE ε4 allele have an increased risk of AD³² and the ε4 allele has been shown to be associated with increased oxidative stress, relative to the ApoE ε2 and ε3 alleles. Hence, the ApoE ε4 allele could increase the mitochondrial oxidative stress and hence the mtDNA somatic mutation rate.

The Aβ peptide, on the other hand, has been proposed that to act as an anti-oxidant defense system to protect neuronal synapses from oxidative damage. However, when Aβ is overproduced it aggregates and becomes a toxic pro-oxidant. Given this scenario, then mtDNA variants in neurons which increase mitochondrial ROS production would stimulate the production of AP. While initially protective, the Aβ would soon aggregate resulting in plaque deposition, increased ROS in the vicinity of the mitochondria-rich synaptic boutons, mtPTP activation, and synaptic loss. Moreover, mutations in the APP or Presenilin complex genes that result in the overproduction of Aβ and its premature aggregation would also increase ROS production, mitochondrial damage, activation of the mtPTP, and synaptic loss.

In conclusion, these data suggest that AD is the product of the accumulation of somatic mtDNA CR mutations which are the product of oxidative damage to the mtDNA. These deleterious mtDNA mutations ultimately result in mitochondrial energy deficiency, increase oxidative stress, activation of the mtPTP, and loss of synaptic connections. Because of the stochastic nature of somatic mtDNA mutations and the pivotal role that the mitochondria play in neuronal energy generation, ROS production, and apoptosis, this mitochondrial hypothesis provides a straightforward explanation for many of the unusual genetic and pathological features of sporadic AD.

### EXAMPLE 2

(Diagnosis of AD in a Pre-Symptomatic Subjects)

Samples of cells (e.g., blood cells, skin fibroblasts, urinary tract epithelial cells, and/or cerebral spinal fluid cells) are obtained from two thirty-five year old human subjects who currently exhibit no symptoms of AD. After the cells have been collected, the DNA is extracted from the cells by well known technique. The DNA from each subject is then subjected to mitochondrial DNA control region amplification and the amplified DNA is then tested for the presence of the homoplasmic 414 and 477 nucleotide variants by direct sequencing for low levels of heteroplasmic 414 and 477 nucleotide mutations by PNA-clamping PCR. If 414 and 477 nucleotide variants are detected by PNA-clamping PCR, the mutant molecules are then cloned and sequenced to confirm the presence of the mutation.

In the first subject, the total number of mutations detected is significantly greater than control, thereby indicating that this subject is likely to develop symptoms of AD later in life.

In the second subject, the total number of mutations detected is not significantly greater than control, thereby indicating that this subject is unlikely to develop symptoms of AD later in life.

### EXAMPLE 3

(Determining Efficacy of AD Treatment)

In this example, an 85 year old patient is diagnosed with AD based on symptomology and clinical presentation. Prior to commencement of therapy, a samples of cells (e.g., blood cells, skin fibroblasts, urinary tract epithelial cells, and/or cerebral spinal fluid cells) is obtained, processed, tested for 414 and 477 nucleotide variants and the mutant molecules are then cloned and sequenced, as as described in Example 2 above, to obtain a quantitative baseline determination of the total number of T4141G, T414C, and T477C mutations. This baseline total number of T4141G, T414C, and T477C mutations is compared to normal control data to confirm that the subject has a greater than expected number of T4141G, T414C, and T477C mutations, which is consistent with the diagnosis of AD. Drug therapy for AD is then commenced. Periodically (e.g., every 6 months) follow-up cell samples are obtained from the subject, processed, tested and cloned in the same manner as the baseline blood sample, thereby obtaining post-treatment quantitative determination(s) of the total number of T4141G, T414C, and T477C mutations. The total number of T4141G, T414C, and T477C mutations determined in each follow-up blood sample is compared to the pre-treatment baseline (and optionally to any earlier follow up blood samples tested) to determine the efficacy of the AD treatment being administered. If the total number of T4141G, T414C, and T477C mutations is seen to decrease significantly as therapy continues, the therapy is deemed to be efficacious in that subject. On the other hand, if the total number of T4141G, T414C, and T477C mutations is seen to remain constant or to increase significantly as therapy continues, the therapy is deemed to be non-efficacious in that subject and adjustments or changes in the therapy may be deemed appropriate.

### EXAMPLE 4

(Diagnosis of Down's Syndrome Dementia)

A 25 year old subject with a confirmed diagnosis of Down's Syndrome currently exhibits no symptoms of dementia. Samples of cells (e.g., blood cells, skin fibroblasts, urinary tract epithelial cells, and/or cerebral spinal fluid cells) are tested for 414 and 477 nucleotide variants and the mutant molecules are then cloned and sequenced, as as described in Example 2 above. If the total number of T4141G, T414C, and T477C mutations is seen to be significantly greater than control, it may be concluded that the subject is likely to develop Down's Syndrome-associated senile dementia later in life. On the other hand, if the total number of T4141G, T414C, and T477C mutations is not significantly greater than normal controls, it may be concluded that the subject is likely to develop Down's Syndrome-associated senile dementia later in life.

In some cases, test kits may be provided for use in performing the cell sample collection, processing, testing and/or cloning in accordance with the present invention. Such test kits may include normal standards (e.g., reference samples) and/or control data for comparison to the test results. Such control data may be provided in the form of a single number, a look-up table, a mechanical or electronic calculator and/or a computer may be programmed to contain such control data and/or to perform comparisons and statistical calculations to determine if the mtDNA CR mutations detected in a particular subject are significantly different from that of a relevant control group.

As used in this patent application, the terms “patient” and “subject” include human as well as other animal patients and subjects that receive therapeutic, preventative, experimental or diagnostic treatment or a human or animal having a disease or being predisposed to a disease.

Although the foregoing invention has been described in detail for purposes of clarity of understanding, it will be obvious that certain modifications may be practised within the scope of the appended claims. All publications and patent documents cited herein are hereby incorporated by reference in their entirety for all purposes to the same extent as if each were so individually denoted.

1. A method for diagnosis of a disorder associated with the development of beta amyloid deposits or fibrils in a human or animal subject or assessing the efficacy of treatment rendered to the subject for such disorder, said method comprising the step of:
A) determining the presence of mtDNA CR mutations.

2. A method according to claim 1, wherein Step A comprises making a qualitative determination that mtDNS CR mutation is or is not present.

3. A method according to claim 1, wherein Step A comprises making a quantitative determination of mtDNS CR mutations.

4. A method according to claim 3 further comprising the step of:
B) comparing a mtDNS CR value obtained by the quantitative determination made in Step A with a control mtDNS CR value to determine whether the subject has significantly more mtDNS CR mutations than control.

5. A method according to claim 3 further comprising the step of:
B) comparing a mtDNS CR value obtained by the quantitative determination made in Step A with a mtDNS CR value representative of subjects who suffer from a disorder associated with the development of beta amyloid deposits or fibrils.

6. A method according to any of claim 1 wherein Step A comprises testing for a T4141G mutation.

7. A method according to any of claim 1 wherein Step A comprises testing for a T414C mutation.

8. A method according to any of claim 1 wherein Step A comprises testing for a T477C mutation.

9. A method according to any of claim 1 wherein Step A comprises testing for a T146C mutation.

10. A method according to any of claim 1 wherein Step A comprises testing for a T152C mutation.

11. A method according to any of claim 1 wherein Step A comprises testing for a A189G mutation.

12. A method according to any of claim 1 wherein Step A comprises testing for a T195C mutation.

13. A method according to claim 1 wherein Step A is carried out at least in part by PNA-clamping PCR.

14. A method according to claim 1 wherein Step A is carried out at least in part by oligonucleotide hybridization.

15. A method according to claim 1 wherein Step A is carried out at least in part by primer extension.

16. A method according to claim 1 wherein Step A is carried out at least in part by restriction digestion.

17. A method according to claim 1 wherein the determination of Step A is made in a specimen of tissue, cells or body fluid selected from the group consisting of:
i. brain tissue;
ii. brain tissue from the frontal cortex;
iii. nervous tissue;
iv. nerve cells
v. blood
vi. blood cells;
vii. urine;
viii. urinary tract cells;
ix. skin;
x. skin cells;
xi. epithelium;
xii. epithelial cells;
xiii. fibroblasts;
xiv. cerebrospinal fluid; and
xv. cells contained in cerebrospinal fluid.

18. A method according to claim 1 wherein the method is carried out for post-symptomatic diagnosis of a disorder in a subject who has begun to exhibit symptoms of that disorder.

19. A method according to claim 1 wherein the method is carried out for pre-symptomatic diagnosis of a disorder in a subject who has not begun to exhibit symptoms of that disorder.

20. A method according to claim 1 wherein the disorder is a neurodegenerative disease.

21. A method according to claim 1 wherein the disorder is Alzheimer's Disease.

22. A method according to claim 1 wherein the disorder is Parkinson's Disease.

23. A method according to claim 1 wherein the disorder is Down's Syndrome-associated dementia.

24. A method according to claim 1 wherein the disorder is a spongiform encephalopathy.

25. A method according to claim 1 wherein the disorder is type II diabetes.

26. A method according to claim 1 wherein the disorder is Creutzfeldt-Jakob disease.

27. A method according to claim 1 wherein the disorder is a Huntington's disease.

28. A method according to claim 1 wherein the disorder is macular degeneration.

29. A method according to claim 1 wherein the disorder is a prion disease.

30. A method according to claim 1 wherein Step A comprises:
obtaining sample cells from the subject;
extracting DNA from the sample cells;
subjecting the extracted DNA to mitochondrial DNA control region amplification;
determining whether homoplasmic 414 and 477 nucleotide variants are present by direct sequencing for heteroplasmic 414 and 477 nucleotide mutations; and
if 414 and 477 nucleotide variants are detected, cloning the mutant molecules and sequencing the clone.

31. (canceled)

32. (canceled)

33. (canceled)

34. (canceled)
